(19) United States
(12) Patent Application Publication
(10) Pub. No.: US 2020/0138282 A1
(43) Pub. Date: May 7, 2020

(54) LARYNGOSCOPY DEVICES AND METHODS

(71) Applicants: Ishay BENURI-SILBIGER, Jerusalem (IL); Shahar MILLIS, Hanna Karkur (IL)

(72) Inventor: Ishay BENURI-SILBIGER, Jerusalem (IL)

(21) Appl. No.: 16/314,916

(22) PCT Filed: Feb. 21, 2017
(86) PCT No.: PCT/IL2017/050215

§ 371 Date: Jan. 3, 2019

(30) Foreign Application Priority Data
Aug. 1, 2016 (IL) 247049

(51) Int. Cl. A61B 1/267; A61M 16/04

(52) U.S. Cl. CPC A61B 1/267; A61M 16/0418

(57) ABSTRACT

Disclosed herein are devices and methods for laryngoscopy. Each of the disclosed devices includes a laryngoscope blade and a looped element associated therewith. The looped element is switchable from a first configuration, wherein it defines an expanded arch mounted on a distal portion of the laryngoscope blade, to a second configuration, wherein it defines a contracted arch mounted on the distal portion. The arches are configured to encompass the epiglottis of a subject when the distal portion of the laryngoscope blade is inserted into the vallecula of the subject during ETI. The expanded arch is configured to facilitate easy insertion therethrough of a distal tip of an elongated member, such as a bougie introducer. Following the insertion of the distal tip, the looped element can be switched to the second configuration. The contracted arch is configured to direct the elongated intubation member towards the laryngeal inlet of the subject.

### TECHNICAL FIELD

The present disclosure generally relates to devices and methods for airway management, particularly in laryngoscopy.

### BACKGROUND

Endotracheal intubation (ETI) is a medical procedure wherein an endotracheal tube is inserted through a subject's mouth or nose into their trachea. ETI is applied across a vast array of age-groups and disease-states to allow oxygen delivery to the lungs in subjects not capable of breathing on their own, in subjects requiring sedation (such as subjects undergoing surgery), in unconscious subjects at risk of aspirating gastric content, and in subjects at risk of airway obstruction (such as subjects suffering from an allergic reaction or burn victims).

Oral ETI is usually performed by positioning the subject in a “sniffing position” (by extending their head and flexing their neck) and introducing through the mouth a laryngoscope blade (typically a Macintosh blade). The blade slides on the subject's tongue until the distal end of the blade reaches the epiglottic vallecula—a depression at the base of the tongue. The laryngoscope's handle is then pulled, lifting the tongue at its base and bringing into alignment the oral axis with the pharyngeal axis, thereby facilitating an unobstructed view of the glottis and creating a pathway for the endotracheal tube.

A variety of causes may complicate ETI, preventing a quick execution thereof. These include: morbid obesity, inability or contraindication to moving the subject's head (e.g. when cervical spine injury is suspected, thereby preventing positioning the subject in the sniffing position), profound oral secretion or perfuse hemorrhage in the oropharyngeal cavity, airway swelling (e.g. due to an allergic reaction or in burn victims), as well as an often steep learning curve experienced by practitioners.

To facilitate rapid ETI in such cases, several aids have been developed. Currently, perhaps the most widely used aid is the elastic endotracheal introducer—the so-called bougie introducer. The bougie introducer is a long stylet with a bent distal tip. The angle of the distal tip acts to facilitate entering the bougie introducer into the laryngeal inlet. When the bougie introducer has been inserted into the laryngeal inlet of the subject, an endotracheal tube is mounted on the bougie introducer and advanced thereon into the trachea, and the bougie introducer is pulled out.

The bougie introducer provides an effective tool for aiding intubation when the epiglottis is visible, even if the vocal cords are not (Grade III Cormack-Lehane view). However, the bougie introducer is unlikely to be of utility when none of the airway is visible (Grade IV Cormack-Lehane view) for reasons elaborated on below.

According to a recent study, a substantial percentage of bougie introduction attempts result in failure. See, for example, http://felipeairway.sites.medinfo.ufl.edu/files/2009/06/the-american-journal-of-emergency-medicine-2005-jabre.pdf. One of the main reasons for bougie introduction failure is esophageal intubation, that is to say, the insertion of the bougie introducer into the esophagus (instead of into the laryngeal inlet). Being narrow, elongated, and flexible, control of the bougie introducer at its tip can be very challenging, particularly when the tongue base obstructs the path into the trachea.

U.S. Pat. No. 8,702,599 to De Domenico discloses an improved laryngoscope that is useful in endotracheal intubation. The laryngoscope includes an inner magnetic element that is situated at an end of the laryngoscope's blade and an outer magnetic element that is positioned on a patient's throat in such a manner that the interaction between the inner and outer elements attracts the end of the blade toward the patient's epiglottic vallecula when the blade is moved into the patient's throat. The blade also includes a magnetic bed located along its longitudinal axis. The magnetic bed is designed to interact with a metallic spiral tube and, by means of this interaction, guide the tube properly in the patient's inner air tract. The blade also includes at least three coplanar elements that able to rotate relative to each other. The adjustment of these elements increases the blade's usefulness for moving the patient's tissues and opening a passage to the patient's trachea.

U.S. Pat. No. 8,366,612 to Rosenthal discloses a method of using a laryngoscope guide including a guiding conduit for advancing an introducer. The method can be used to intubate a subject by positioning the laryngoscope guide in an airway; advancing the introducer in the conduit until an end of the introducer enters the glottis, while viewing images of the introducer end; removing the laryngoscope guide with the guide conduit from the introducer and from the airway; guiding a tube with the introducer until an end of the tube enters the glottis; and removing the introducer from the tube with the end of the tube remaining in the glottis, and in particular, in the trachea, to establish an airway. The guide and method can enable a healthcare professional to intubate a subject where neck mobility is an issue, where an airway is difficult, and/or where a subject is obese.

### SUMMARY

Aspects of the disclosure, in some embodiments thereof, relate to devices and methods for controlling and maneuvering instruments in airway management. More specifically, aspects of the disclosure, in some embodiments thereof, relate to devices and methods for laryngoscopy.

As explained above, there is a need for ETI devices and methods facilitating rapid and accurate ETI. Specifically, there is a need for ETI devices and methods facilitating rapid and accurate ETI in Grade III and particularly Grade IV Cormack-Lehane view conditions. The present application, according to some of the embodiments disclosed herein, addresses this need.

According to an aspect of some embodiments, there is provided a laryngoscopy device including:
a laryngoscope blade, being elongated, having a blade distal portion, a blade first edge, and a blade second edge, opposite to the blade first edge; and
a looped element, switchable at least from a first configuration to a second configuration.

In the first configuration, and in the second configuration, the looped element defines an expanded arch and a contracted arch, respectively. The expanded arch has an expanded arch first arm and an expanded arch second arm, joined at an expanded arch top. The contracted arch has a contracted arch first arm and a contracted arch second arm, joined at a contracted arch top.

The expanded arch top is positioned superiorly relative to the blade distal portion. The expanded arch first arm at least partially extends laterally from about the blade first edge and the expanded arch second arm at least partially extends laterally from about the blade second edge.

According to some embodiments, the looped element is continuously switchable from the first configuration to the second configuration, across a range of mid-configurations, defining a respective range of increasingly smaller arches, ranging from the expanded arch to the contracted arch.

According to some embodiments, the looped element is additionally switchable from the second configuration to the first configuration.

According to some embodiments, the looped element is continuously switchable from the second configuration to the first configuration.

According to some embodiments, the contracted arch top is positioned superiorly relative to the blade distal portion.

According to some embodiments, the blade distal portion is curved and the expanded arch and the contracted arch are each configured such as (i) to encompass the epiglottis of a subject when the laryngoscopy device is inserted into the oral cavity of the subject with the blade distal portion being inserted into the vallecula of the subject, and (ii) to simultaneously facilitate guiding of an elongated intubation member through the expanded arch and through the contracted arch, respectively.

According to some embodiments, the elongated intubation member is a bougie introducer, a stylet, or an endotracheal tube.

According to some embodiments, there is provided a laryngoscope including the laryngoscopy blade and a handle, wherein the laryngoscope blade is mechanically connected to the handle.

According to an aspect of some embodiments, there is provided a laryngoscopy device including:
a laryngoscope blade, being elongated, having a blade distal portion;
a looped element, switchable at least from a first configuration to a second configuration; and
a mounting mechanism.

In the first configuration and in the second configuration the looped element respectively defines an expanded arch, having an expanded arch base, and a contracted arch, having a contracted arch base.

The mounting mechanism directly or indirectly supports (for example, mechanically supports, magnetically supports, or both) the expanded arch and the contracted arch in the first configuration and in the second configuration, respectively.

The mounting mechanism substantially restricts the expanded arch base and the contracted arch base to a mounting site on the blade distal portion.

According to some embodiments, an arch top of the expanded arch is positioned superiorly relative to the blade distal portion.

According to some embodiments, the expanded arch base and the contracted arch base are controllably shiftable from the mounting site to at least one other mounting site on the blade distal portion.

According to some embodiments, the expanded arch is wider than the blade distal portion.

According to an aspect of some embodiments, there is provided a method for performing laryngoscopy on a subject. The method includes the steps of:
providing any one of the laryngoscopy devices and an elongated intubation member;
introducing the laryngoscopy device into the oral cavity of the subject with the looped element being in the first configuration;
maneuvering the laryngoscope blade such as to insert the blade distal portion into the vallecula of the subject, thereby encompassing the epiglottis of the subject with the expanded arch;
introducing the elongated intubation member into the oral cavity and distally advancing the elongated intubation member until a member distal tip thereof passes through the expanded arch and is proximally positioned relative to the corniculate tubercle of the subject;
switching from the first configuration to the second configuration thereby directing the member distal tip towards the laryngeal inlet of the subject; and
distally advancing the member distal tip into the laryngeal inlet of the subject.

According to some embodiments, the elongated intubation member is a bougie introducer, and the method further includes, following the step of distally advancing the member distal tip into the laryngeal inlet of the subject, a step of mounting an endotracheal tube on the bougie introducer.

According to some embodiments, the elongated intubation member is an endotracheal tube.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a laryngoscopy device, including a laryngoscope blade and looped element in a first configuration (solid curve) and in a second configuration (dashed curve), according to some embodiments;

FIG. 2A schematically illustrates a laryngoscope including the laryngoscopy device of FIG. 1 and a handle, the laryngoscopy device being partially inserted into the oral cavity of a subject, with the looped element being in the first configuration and encompassing the epiglottis of the subject, and with a bougie introducer inserted through the looped element, according to some embodiments;

FIG. 2B schematically illustrates the laryngoscope of FIG. 2A, the laryngoscopy device being partially inserted into the oral cavity of the subject, with the looped element being in the second configuration, encompassing the epiglottis of the subject, and directing the bougie introducer towards the laryngeal inlet of the subject, according to some embodiments;

FIG. 3 schematically illustrates a laryngoscopy device, according to some embodiments;

FIG. 4A schematically illustrates a laryngoscope, according to some embodiments;

FIG. 4B schematically illustrates a back-view of a hand grip of a guiding assembly of the laryngoscope of FIG. 4A, according to some embodiments;

FIG. 5 schematically illustrates a laryngoscopy device, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

As used herein, in accordance with some embodiments, a “laryngoscope blade” may refer to a single piece (integrally formed) blade, as well as to a non-integrally formed blade including, for example, a blade portion and one or more blade extensions mounted thereon or connected thereto. In particular, in accordance with some embodiments, a “laryngoscope blade” may refer to a blade portion and to an extension mounted thereon, for example, on a distal tip of the blade portion.

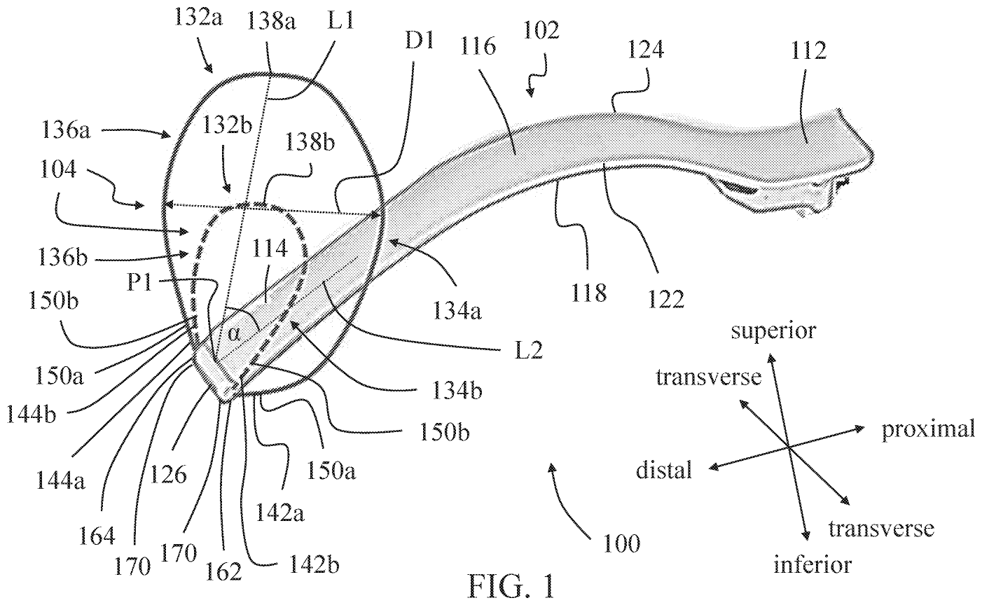

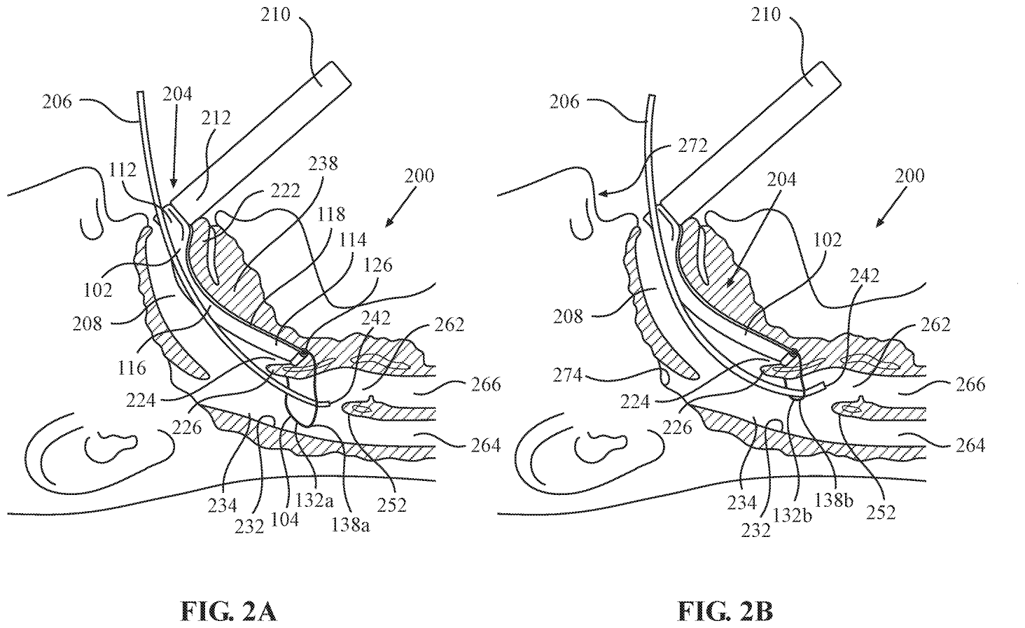

FIG. 1 schematically depicts a laryngoscopy device 100, including a laryngoscope blade 102 and a looped element 104, according to some embodiments of the present disclosure. Laryngoscope blade 102 is elongated and includes a blade proximal portion 112 and a blade distal portion 114. Laryngoscope blade 102 further includes a blade superior surface 116, a blade inferior surface 118, and opposite lateral edges: a blade first edge 122, and a blade second edge 124. A blade tip 126 is defined by the distal edge of blade distal portion 114. According to some embodiments, laryngoscope blade 102 may be mountable on a handle, as depicted, for example, in FIGS. 2A-2B.

It is noted that blade first edge 122 and blade second edge 124 need not be symmetric. For example, in some embodiments, blade first edge 122 may be thin, while blade second edge 124 may be thick. In particular, according to some embodiments, blade second edge 124 (or blade first edge 122) includes a sidewall mounted at right angles to blade superior surface 116 (i.e. perpendicularly to the transverse direction). The sidewall is used to laterally move the tongue during ETI, and is a standard feature in the art of laryngoscopes. For ease of presentation, the sidewall is not depicted in the Figures.

Looped element 104 is switchable from a first configuration to a second configuration. In the first configuration, looped element 104 defines an expanded arch 132a (marked by a solid curve). In the second configuration looped element 104 defines a contracted arch 132b (marked by a dashed curve). Expanded arch 132a extends superiorly relative to blade superior surface 116 at blade distal portion 114. Expanded arch 132a includes an expanded arch first arm 134a and an expanded arch second arm 136a, which are of substantially equal length. Expanded arch first arm 134a and expanded arch second arm 136a are joined at an expanded arch top 138a.

Expanded arch top 138a is positioned superiorly relative to blade distal portion 114, at the most superior position along expanded arch 132a. An expanded arch first end 142a, defined by the inferior end of expanded arch first arm 134a, projects substantially laterally from, or approximately from, blade first edge 122 at blade distal portion 114 (i.e. away from blade distal portion 114). In some embodiments, expanded arch first end 142a additionally projects in the superior direction. An expanded arch second end 144a, defined by the inferior end of expanded arch second arm 136a, projects substantially laterally from, or approximately from, blade second edge 124 at blade distal portion 114 (i.e. away from blade distal portion 114). In some embodiments, expanded arch second end 144a additionally projects in the superior direction.

According to some embodiments, expanded arch 132a is substantially shaped as a horseshoe, and is mounted substantially at right angles to blade superior surface 116, with a first and second horse shoe legs mounted approximately at blade first edge 122 and blade second edge 124, respectively. That is to say, expanded arch first arm 134a extends laterally and superiorly from blade first edge 122 before starting to curve in the medial direction towards expanded arch top 138a. Expanded arch second arm 136a extends laterally and superiorly from blade second edge 124 before starting to curve in the medial direction towards expanded arch top 138a.

A line L1 is defined as the straight line extending from expanded arch top 138a to a point P1 defined midway between blade first edge 122 and blade second edge 124 at blade tip 126. A line L2 is defined as the line extending from point P1 in the proximal direction along blade superior surface 116 and passing midway between blade first edge 122 and blade second edge 124 at blade distal portion 114. Lines L1 and L2 define an extension angle α of expanded arch 132a. In some embodiments, extension angle α is selected to be in the range of about 60° to about 160°, or between about 80° to about 160°, or between about 70° to about 130°, or even between about 75° to about 115°. In some embodiments, extension angle α may be controllably adjusted, from about 60° to about 160°, or from about 80° to about 160°, or from about 70° to about 130°, or even from about 75° to about 115°.

Contracted arch 132b extends superiorly from, or approximately from, blade superior surface 116 at blade distal portion 114. Contracted arch 132b includes a contracted arch first arm 134b and a contracted arch second arm 136b, which are of substantially equal length. Contracted arch first arm 134b and contracted arch second arm 136b are joined at a contracted arch top 138b. According to some embodiments, contracted arch top 138b is positioned superiorly relative to blade distal portion 114, at the most superior position along contracted arch 132b. According to some embodiments, contracted arch 132b does not substantially extend superiorly from blade superior surface 116, substantially extending instead in the distal direction, as elaborated on below. According to some embodiments, contracted arch 132b is substantially shaped as a horseshoe. Contracted arch 132b is smaller than expanded arch 132a.

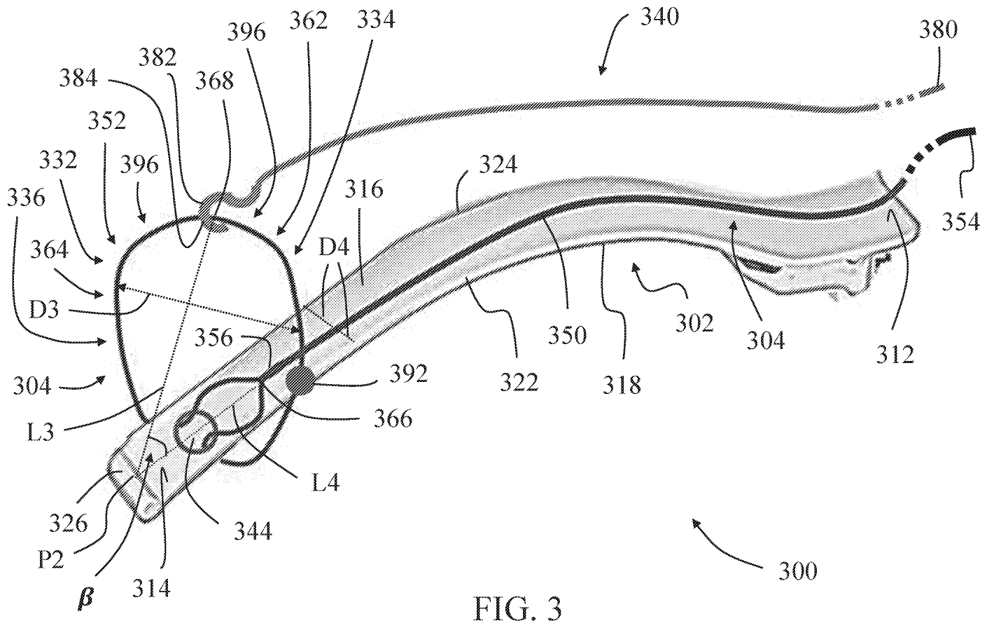

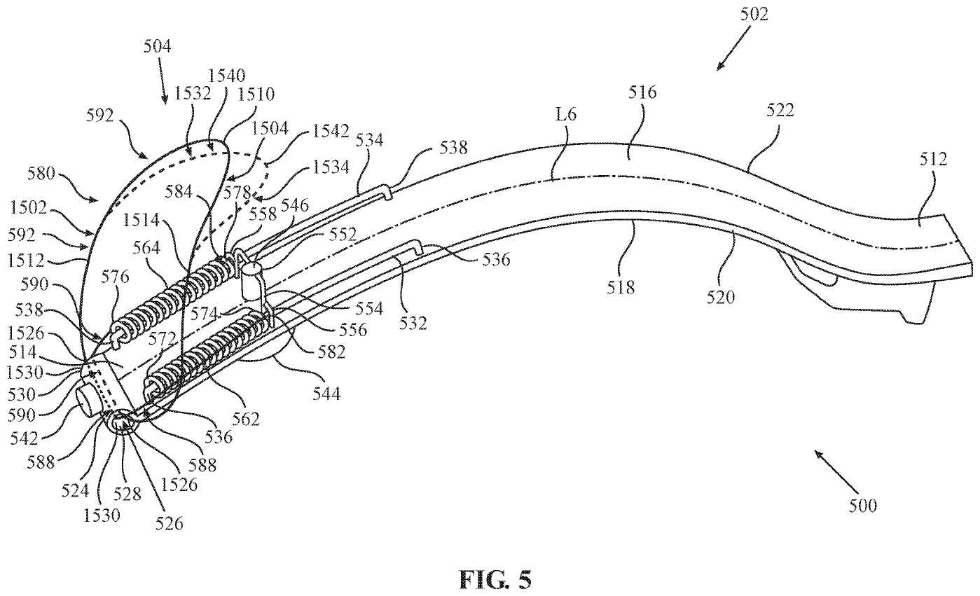

According to some embodiments, looped element 104 is additionally switchable from the second configuration to the first configuration. According to some embodiments, looped element 104 is continuously switchable from the first configuration to the second configuration. That is to say, looped element 104 can be switched across a continuous range of mid-configurations characterized by increasingly smaller arches ranging from expanded arch 132a to contracted arch 132b Similarly, according to some embodiments, looped element 104 is continuously switchable from the second configuration to the first configuration. Mechanisms to achieve and sustain expanded arch 132a and contracted arch 132b superior extension, as well as mechanisms for switching from the first configuration to the second configuration, and/or for continuously switching between the configurations, are elaborated on below in the description of FIGS. 3-5. According to some embodiments, looped element 104 is pre-shaped, thereby maintaining the superior extension of expanded arch 132a and contracted arch 132b, in an essentially similar manner to that described in the description of the laryngoscopy device of FIG. 5. According to some embodiments, looped element 104 is made of flexible and resilient material. For example, looped element 104 may include a stainless steel wire, a wire made of a metal alloy, such as Nitinol, a wire made of flexible polymeric material, such as nylon, or a wire made of a combination of metal and polymeric material. The material can be coated by a non-stick material such as Teflon™ to reduce friction. The material can be or include a rare earth magnet to facilitate contact with metal objects (for example a magnetic plate glued to the tip of an endotracheal introducer or tube). In some embodiments, a portion of looped element 104 consisting of expanded arch 132a may be made of a single stranded wire or a braided wire. In some embodiments the looped element includes chemiluminescence (glow in the dark) material.

In some embodiments, different portions of looped element 104 are made of different materials and have substantially different shapes. For example, in some embodiments the arms of the looped element are made of a plastic wire, while the top of the looped element is made of metal alloy and shaped as a cuboid plate or prism. The arms may be made of a wire that branches in the superior direction to several wires that are connected to a mesh-like structure resembling a rectangular parachute canopy. In some embodiments the canopy angulation can be manipulated by shortening of a single or several of the branched wires. In some embodiments the mesh acts as a net that can entrap and restrict advancement of an object; upon a change in configuration of the object or the net-like structure, the object can be released and advanced further.

In some embodiments, the looped element is composed of separate elements, which are detachable from one another, for example, by mechanical or electrical mechanism, thus allowing to disconnect and pull apart the two arms of an arched formation of the looped element. For example, a grasping forceps commonly used for endoscopic procedures may embody one arm of the looped element, grasping an end of the second arm of the looped element with locked jaws; the resultant arched formation may be dismantled when the locking of the grasper is released and the jaws open. In another example, one arm of the looped element is attached to end of the second arch by an electromagnetic element that can controllably turned off or produce repulsing force. In some embodiments, one arm of the looped element is detached from the base of the arch by similar mechanism (for example, mechanical and/or electrical)

In some embodiments, the looped element may be multi-functional and also be formed of hollow tubing and holes with properties of a suction device. In some embodiments, the looped element includes a mirror that provides the practitioner an additional angle of view of the larynx. In some embodiments, the looped element includes a fiber optic light source, optic fiber transmitting images and/or miniature camera. In some embodiments, the looped element includes elements of electronic sensor or sensors (for example, an infrared or a metal detector) that recognize proximity of an object, enabling blind intubation. In some embodiments, the looped element is made of hollow tubing and an inflatable balloon. Inflation of the balloon may change the arch from one configuration to a more space occupying configuration, obstructing the path to the esophagus and shifting an inserted object toward the larynx

In some embodiments, the arms of the looped element can be placed in the lateral aspect of the blade by parallel conduits running alongside the edges of the blade; another option is that of a single conduit running along the blade and splitting, proximally to the mounting site of the arch, into two branches (‘Y’ shaped or ‘T’ shaped) respectively directed (laterally) to the two edges of the blade.

In some embodiments, expanded arch top 138a may be positioned, for example, at a distance of about 3 cm to about 6 cm from blade distal portion 114. Expanded arch 132a may have a width Dl measuring, for example, between about 2.5 cm to about 4.5 cm. Contracted arch top 138b may be positioned, for example, at a distance of about 1.5 cm to about 3 cm from blade distal portion 114. Contracted arch 132b may have a width (not indicated) measuring, for example, between about 1 cm to about 2 cm.

More specifically, a size of expanded arch 132a is selected such as to facilitate easy insertion therethrough of a bougie introducer, a stylet, or even an endotracheal tube, when laryngoscopy device 100 is inserted into the oral cavity of a subject, as elaborated on below. A size of contracted arch 132b is selected such as to direct the bougie introducer, the stylet, and in some embodiments the endotracheal tube, into the laryngeal inlet of the subject, when laryngoscopy device 100 is inserted into the oral cavity of a subject, as elaborated on below. The sizes of expanded arch 132a and contracted arch 132b may vary with the size of laryngoscope blade 102. In particular, the size of expanded arch 132a, and in some embodiments also the size of second arch 132b, will depend on physical characteristics of the subject, e.g. height, weight, gender, with the sizes of expanded arch 132a typically being larger for adults than for children, for adult males than for adult females, etc.

An expanded arch base 150a is defined by expanded arch first end 142a and expanded arch second end 144a. A contracted arch base 150b is defined by a contracted arch first end 142b and a contracted arch second end 144b, that is to say, the inferior ends of contracted arch first arm 134b and contracted arch second arm 136b, respectively.

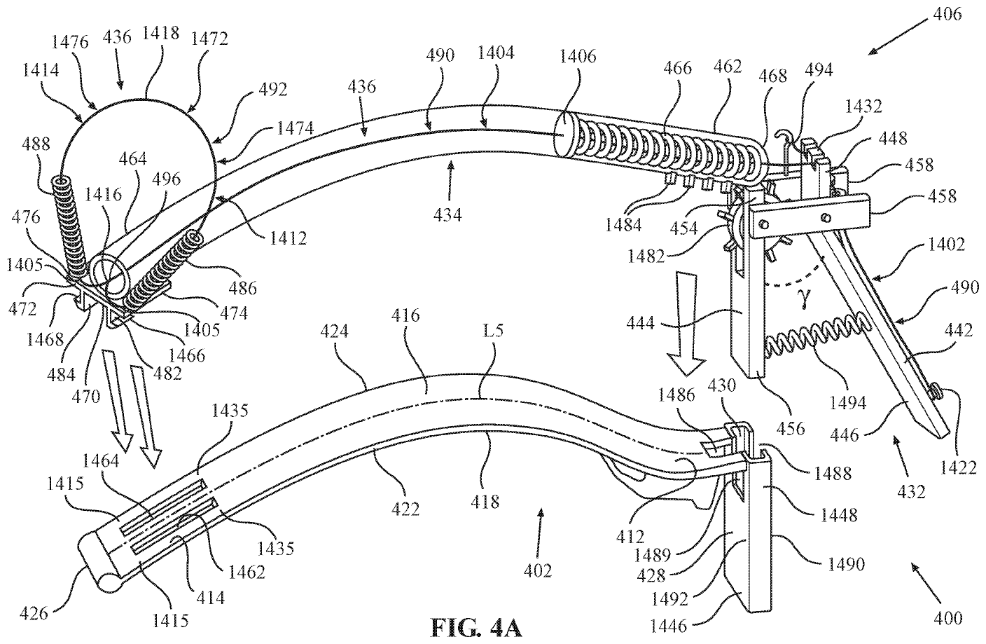

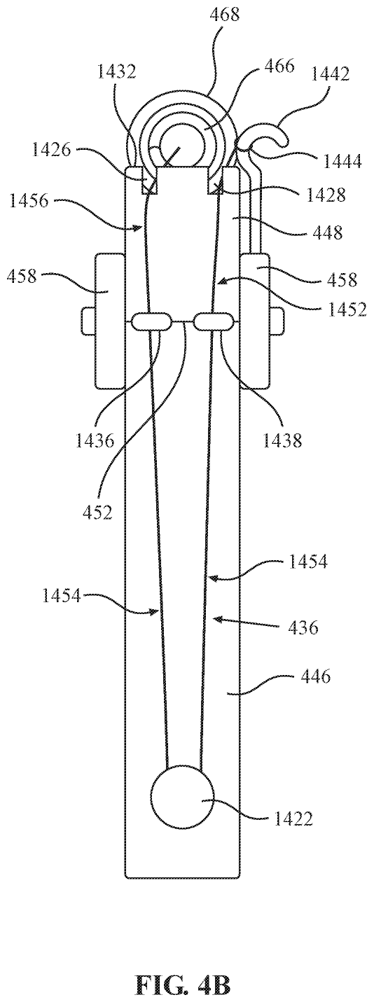

In some embodiments, laryngoscopy device 100 includes a mounting mechanism (not shown in FIG. 1, see, for example, FIGS. 4A and 5). The mounting mechanism supports arches 132a and 132b and further substantially restricts expanded arch base 150a to a first mounting location 162 on blade first edge 122, and contracted arch base 150b to a second mounting location 164 on blade second edge 124. According to some embodiments, first mounting location 162 and second mounting location 164 are each positioned proximally relative to blade tip 126, at a distance selected from the range of about 0 cm to about 3 cm from blade tip 126, or from about 1 cm to about 2.5 cm therefrom. A mounting site 170 consists of mounting locations 162 and 164.

In some embodiments, arch bases 150a and 150b are controllably movable from mounting site 170 to a second mounting site (not shown in FIG. 1, see, for example, FIG. 4A) on blade distal portion 114. The second mounting site is longitudinally positioned (in some embodiments proximally positioned and in some embodiments distally positioned) relative to mounting site 170, thereby facilitating longitudinally moving expanded arch 132a in order to accommodate a variety of epiglottic anatomy.

FIGS. 2A-2B schematically depict laryngoscopy device 100 in operation. FIGS. 2A-2B depict an embodiment of laryngoscope blade 102 wherein blade distal portion 114 curves in the anterior direction (that is to say, in the inferior direction per the directions defined in FIG. 1), similarly to a Macintosh blade. In some embodiments, laryngoscope blade 102 is a Macintosh blade. However, the skilled person will understand that embodiments of laryngoscope blade 102 are not limited to curved blades. In particular, laryngoscope blade 102 may also be embodied as a Miller blade (i.e. with blade distal portion 114 being straight), as elaborated on below.

FIGS. 2A-2B schematically depict the mouth and upper throat region 200 of a subject with a laryngoscope 204 and a bougie introducer 206 inserted into the oral cavity 208 of the subject. Laryngoscope 204 includes laryngoscopy device 100 and a handle 210. Blade proximal portion 112 is (mechanically) connected to a handle top 212 of handle 210. In some embodiments, blade proximal portion 112 is mounted on handle top 212 in an ‘r’-like configuration (per the directions defined in FIG. 1).

In operation, laryngoscopy device 100 is inserted into oral cavity 208, with looped element 104 being in the first configuration. Laryngoscope blade 102 is maneuvered such that blade inferior surface 118 is slid on the tongue 222 of the subject, and blade distal portion 114 is inserted into the vallecula 224 of the subject. As blade distal portion 114 is inserted into vallecula 224, expanded arch 132a comes to encompass the epiglottis 226 of the subject, with expanded arch top 138a positioned proximately to, or resting against, the laryngopharyngeal posterior wall 232 (that is to say, the posterior wall of the laryngopharynx 234 of the subject), as shown in FIG. 2A. Handle 210 is then directed anteriorly and caudally (distally) with respect to the subject, thereby lifting the base of the tongue 238 of the subject, and bringing into alignment the oral axis of the subject with their pharyngeal axis (both not indicated), as is standard in the art of laryngoscopy.

Next, bougie introducer 206 is introduced into oral cavity 208 and distally advanced until a distal tip 242 of bougie introducer 206 passes through expanded arch 132a, such as to be (distally) positioned beyond expanded arch 132a, for example, by about 1 cm, or by about 2 cm with distal tip 242 being positioned proximally relative to the corniculate tubercle 252 of the of the subject (whereat the laryngeal inlet 262 of the subject and the esophagus 264 of the subject meet).

After distal tip 242 has been advanced through expanded arch 132a, looped element 104 is switched from the first configuration to the second configuration, which is depicted in FIG. 2B. Contracted arch top 138b is positioned more proximately to epiglottis 226 than to laryngopharyngeal posterior wall 232. As bougie introducer 206 is distally advanced farther, contracted arch 132b directs distal tip 242 into laryngeal inlet 262. Next an endotracheal tube is mounted on bougie introducer 206 and advanced thereon into laryngeal inlet 262 and therefrom into the trachea 266 of the subject.

In some embodiments, wherein an elongated, large caliber instrument (such as an endotracheal tube) rather than a small caliber instrument (such as a stylet or a bougie introducer, such as bougie introducer 206) is inserted through contracted arch 132b (without being mounted on a bougie introducer) contracted arch top 138b may be positioned more proximately to laryngopharyngeal posterior wall 232 than to epiglottis 226. In some embodiments, expanded arch 132a size is sufficiently big to admit insertion therethrough of an endotracheal tube, while contracted arch 132b may be too small to admit insertion therethrough of an endotracheal tube (but nevertheless sufficiently big to admit insertion therethrough of a bougie introducer, such as bougie introducer 206).

According to some embodiments, expanded arch 132a does not substantially extend superiorly from blade superior surface 116, substantially extending instead in the distal direction. Before inserting the laryngoscope device into the oral cavity, the arch is folded (e.g. manually folded by the person performing the ETI) in the proximal direction (that is to say, the arch top is made to point proximally) During the insertion, the arch will be kept folded, being prevented from unfolding due to the comparably narrow dimensions of the oropharynx and the laryngopharynx. Specifically, so long as the arch is sufficiently long (e.g. about 10 cm) the arch top will rest against the posterior wall of the pharynx. Once the blade tip is inserted into the vallecula, the arch is contracted and the arch top detaches from the posterior wall of the pharynx. The arch top then partially unfolds and comes to encompass the epiglottis. The lateral ligaments of the epiglottis prevent a full unfolding of the arch (that is to say, prevent the arch from returning to the original orientation thereof in the distal direction), thereby leading to an encompassing of the epiglottis with a desired angulation of the arch, through which intubation devices can be inserted.

According to some embodiments, expanded arch 132a is substantially as wide as blade distal portion 114. That is to say, expanded arch first arm 134a and expanded arch second arm 136a do not laterally project from blade first edge 122 and blade second edge 124, respectively. According to some embodiments, blade distal portion 114 is wider than expanded arch 132a.

According to some embodiments, laryngoscopy device 100 may include one or more additional looped elements (not shown) similar to looped element 104. For instance, beyond looped element 104, a second looped element (not shown) may be mounted on blade distal portion 114. The second looped element may be positioned proximally relative to looped element 104. For example, looped element 104 may be mounted on blade tip 126, while the second looped element may be mounted on blade distal portion 114, at a distance (as measured along line L2) of about 1 cm or about 2 cm from looped element 104. Similarly to looped element 104, the second looped element is also switchable between at least two configurations, which define respective expanded and contracted arches. In particular, the second looped element's expanded arch may be bigger or smaller than expanded arch 132a and differently shaped and/or oriented. Similarly, the second looped element's contracted arch may be bigger or smaller than contracted arch 132b and differently shaped and/or oriented.

In some embodiments, the second looped element is associated with the looped element 104. For example, the second looped element and looped element 104 may be connected by a thin metal net or a thin plastic film. The second looped element may help in guiding bougie introducer 206 (or other types of elongated intubation members, such an endotracheal tube) into laryngeal inlet 262 during ETI, by providing better support and the possibility of angulation (for example, when the second looped element's expanded arch is bigger than expanded arch 132a).

According to some embodiments, laryngoscope device 100 can be used for nasal ETI. In operation, laryngoscope blade 102 is inserted into oral cavity 208 when looped element 104 is in the first configuration. Laryngoscope blade 102 is maneuvered such that blade inferior surface 118 is slid on the tongue 222 of the subject, and blade distal portion 114 is inserted into the vallecula 224 of the subject. As blade distal portion 114 is inserted into vallecula 224, expanded arch 132a comes to encompass the epiglottis 226 of the subject, essentially as described above for oral ETI. A nasal endotracheal tube (not shown) is inserted into a nostril 272 of the subject. The nasal endotracheal tube is distally advanced through the nasal passage (not indicated) until a distal tip thereof exits the nasal passage into the nasopharynx 274 of the subject. The nasal endotracheal tube is distally advanced farther until the distal tip passes through expanded arch 132a and is positioned proximally relative to conrniculate tubercle 252. Looped element 104 is then switched from the first configuration to the second configuration, thereby directing the distal tip of the elongated member towards laryngeal inlet 262, essentially as described above in the description of FIGS. 2A-2B.

According to some embodiments, blade distal portion 114 is straight (e.g. laryngoscope blade 102 is a Miller-type blade). In operation, laryngoscope blade 102 is inserted into oral cavity 208 when looped element 104 is in the first configuration. Laryngoscope blade 102 is then maneuvered such that blade inferior portion 118 anteriorly presses against epiglottis 226, thereby pushing epiglottis 226 against base of the tongue 238. An elongated member, such as bougie introducer 206, is next inserted into oral cavity 208 and distally advanced until a distal tip thereof passes through expanded arch 132a and is positioned proximally relative to conrniculate tubercle 252. Looped element 104 is then switched from the first configuration to the second configuration, thereby directing the distal tip towards laryngeal inlet 262, essentially as described above in the description of FIGS. 2A-2B.

FIG. 3 schematically depicts a laryngoscopy device 300, including a laryngoscope blade 302, a looped element 304, and a flexible rod 340, according to some embodiments of the present disclosure. Laryngoscope blade 302 is elongated and includes a blade proximal portion 312 and a blade distal portion 314 (per the directions defined in FIG. 1 which also apply for FIG. 3). Laryngoscope blade 302 further includes a blade superior surface 316, a blade inferior surface 318, and opposite lateral edges: a blade first edge 322, and a blade second edge 324. A blade tip 326 is defined by the distal edge of blade distal portion 314. Laryngoscope blade 302 is essentially similar to laryngoscope blade 102 and further includes a bore 344. Bore 344 extends from blade inferior surface 318 to blade superior surface 316 at blade distal portion 314. According to some embodiments, laryngoscope blade 302 can be mounted on a handle, such as handle 210, essentially as depicted, for example, in FIGS. 2A-2B.

Looped element 304 includes an elongated wire 350 and a loop 352. Elongated wire 350 extends from a wire proximal end 354 to a wire distal end 356. Loop 352 includes a loop first arm 362 and a loop second arm 364, which are of substantially equal lengths. Each of first loop arm 362 and second loop arm 364 extends from a loop base 366 to a loop top 368. That is to say, loop first arm 362 and loop second arm 364 are joined both at loop base 366 and at loop top 368. Loop top 368 is positioned superiorly relative to blade distal portion 314, at the most superior position along loop 352. Elongated wire 350 and loop 352 are mechanically connected at wire distal end 356 and at loop base 366, respectively. In some embodiments, elongated wire 350 and loop 352 are integrally formed of a single wire, which is flexible, being shaped into a loop at a distal portion thereof. In some embodiments, loop 352 is made of a different material than elongated wire 350. For example, elongated wire 350 may be metallic and loop 352 may be made of plastic. Or, for example, elongated wire 350 may be made of one type of metal or alloy and loop 352 may be made of another type of metal or alloy. In some embodiments, loop base 366 is fused onto wire distal end 356. In some embodiments, loop base 366 is glued onto wire distal end 356.

Loop 352 includes an arched portion 332. Arched portion 332 is similar to expanded arch 132a, being substantially shaped as a horseshoe and extending superiorly relative to blade superior surface 316 at blade distal portion 314. Arched portion 332 includes an arched portion first arm 334 and an arched portion second arm 336. Arched portion first arm 334 consists of a portion of loop first arm 362, which extends from, or approximately from, blade first edge 322 to loop top 368. Arched portion second arm 336 consists of a portion of loop second arm 364, which extends from, or approximately from, blade second edge 324 to loop top 368.

Similarly to looped element 104, looped element 304 is switchable from a first configuration to a second configuration. In the first configuration, arched portion 332 defines an expanded arch (not shown) similar to expanded arch 132a. In the second configuration, arched portion 332 defines a contracted arch (not shown) similar to contracted arch 132b. Further, looped element 304 is continuously switchable from the first configuration to the second configuration, defining a range of mid-configurations with increasingly smaller arches, as elaborated on below. FIG. 3 depicts one of these mid-configurations (i.e. in FIG. 3 arched portion 332 is not fully expanded or contracted). According to some embodiments, arched portion 332 is wider than blade distal portion 314. That is to say, a straight line D3, which is defined as the longest line extending in the transverse direction from arched portion first arm 334 to arched portion second arm 336, is longer than a line D4, which is defined as extending in the transverse direction from blade first edge 322 to blade second edge 324 at blade distal portion 314. According to some embodiments, arched portion 332 is substantially as wide as blade distal portion 314, or even narrower than blade distal portion 314.

It is noted that in some embodiments, wherein laryngoscope blade 302 includes a sidewall, as described in the description of laryngoscopy device 100, and wherein the sidewall distally extends beyond bore 344, arched portion 332 encompasses the sidewall. In other embodiments, wherein laryngoscope blade 302 includes the sidewall, arched portion second arm 336 extends through a hole in the sidewall; the hole being positioned proximately to blade superior surface 316.

Flexible rod 340 extends from a rod proximal end 380 to a (distal) rod tip 382. Looped element 304 and flexible rod 340 are connected at loop top 368 and at rod tip 382, respectively. Rod tip 382 holds loop 352, thereby sustaining, or helping to sustain, arched portion 332 superior extension. In some embodiments, loop top 368 is fused onto rod tip 382. In some embodiments, loop top 368 is glued onto rod tip 382. In some embodiments, looped element 304 and flexible rod 340 connection is detachable, i.e. looped element 304 and flexible rod 340 can be connected and later disconnected and vice-versa. An example of such a detachable connection is depicted in FIG. 3, wherein rod tip 382 is embodied in the form of a hook 384. Hook 384 holds loop 352 at loop top 368. In some embodiments, rod 340 also has properties of an endotracheal introducer. In such embodiment, the rod can be detached from the loop and advanced into the larynx and act as an endotracheal introducer. In some embodiments, hook 384 can be replaced by a circular element that holds loop 352 at loop top 368; the circular element contains closing mechanism that resembles a circular earrings spring closure. The circular element can be opened or closed by a string that extends from the distal end of the rod/endotracheal introducer. In some embodiments hook 384 can be replaced by an element resembling an endoscopic grasping tool that holds loop 352 at loop top 368.

A line L3 is defined by the straight line extending from loop top 368 to a point P2 defined midway between blade first edge 322 and blade second edge 324 at blade tip 326. A line L4 is defined as the line extending from point P2 in the proximal direction along blade superior surface 316 and passing midway between blade first edge 322 and blade second edge 324 at blade distal portion 314. Lines L3 and L4 define an extension angle β. By distally pushing flexible rod 340, extension angle β may be increased (i.e. loop top 368 is distally pushed). By proximally pulling flexible rod 340 (e.g. by proximally pulling on rod proximal end 380), extension angle β may be decreased (i.e. loop top 368 is proximally pulled). In some embodiments, laryngoscopy device 300 includes a string in place of flexible rod 340, and looped element 304 and the string are connected at loop top 368 and at the distal end of the string, respectively. In such embodiments extension angle β may only be decreased (since the string may only be pulled but not pushed).

It is noted that laryngoscopy device 300 can be easily assembled, e.g. by a doctor or a nurse, from laryngoscope blade 302, looped element 304, and flexible rod 340. For example, wire proximal end 354 may be threaded through bore 344 from blade inferior surface 318 to blade superior surface 316. Wire proximal end 354 may then be proximally pulled, until loop base 366 approaches/reaches/passes bore 344. Loop 352 is next pulled superiorly and brought over blade tip 326. Loop 352 is then attached to hook 384 at loop top 368. In embodiments wherein looped element 304 and flexible rod 340 are integrally formed, laryngoscopy device 300 may be assembled by passing flexible rod through bore 344 from blade superior surface 316 to blade inferior surface 318. When a sufficiently large portion of loop 352 has been threaded through bore 344, flexible rod 340 may be maneuvered such as to superiorly pull loop 352 and to bring loop 352 over blade tip 326.

To switch from the first configuration to the second configuration, elongated wire 350 is pulled, e.g. at wire proximal end 354, in the proximal direction, thereby contracting arched portion 332. More specifically, loop base 366 is pulled in the proximal direction, arched portion first arm 362 and arched portion second arm 364 shorten, and loop top 368 is pulled in the inferior direction (causing, in some embodiments, extension angle (3 to decrease). Similarly, looped element 304 can be switched from the first configuration to any one of the mid-configurations, or from one of the mid-configurations to another one defining a smaller arch than the former.

In some embodiments, loop first arm 362 includes a bead 392, mounted thereon. Bead 392 has a diameter greater than that of bore 344. Bead 392 is positioned on loop first arm 362 such as to block bore 344 when looped element 304 is in the second configuration, thereby preventing any further pulling of loop 352 through bore 344 and maintaining the second configuration. Further, in some embodiments, bead 392 may rotate about loop first arm 362 at bead 392 mounting position. During ETI, bead 392 rotation may help to decrease friction between loop 352 and an elongated member passed therethrough, such as a thick endotracheal tube. In these embodiments, bead 392 may have a diameter smaller than that of bore 344. In some embodiments, two or more beads are mounted on loop first arm 362 and/or loop second arm 364. In some embodiments, a shock-absorbing bead (not shown), that is to say, a bead made of a material with shock absorbent properties, such as rubber, can be mounted on loop 352 at loop top 368. The shock-absorbing bead, being mounted on a portion of loop 352, which may induce the most substantial anteriorly directed force on an instrument inserted during ETI (e.g. bougie introducer 206) as compared to any other portion of loop 352, may help to prevent abrupt jerking movements of the instrument's tip towards laryngeal inlet 262, thereby preventing possible damage to surrounding tissue.

In some embodiments, loop 352 is made of a material having little or substantially no flexibility and rigidity, such as a string. In such embodiments, arched portion 332 superior extension may be maintained exclusively by flexible rod 340.

In some embodiments, loop top 368 consists of a triangularly shaped projection, which may help prevent deformation of loop 352 when inserting laryngoscope blade 302 into oral cavity 208 during ETI.

Laryngoscopy device 300 operation is similar to that of laryngoscope 204. In operation, laryngoscopy device 300 is inserted into oral cavity 208 with loop top 368 attached onto rod tip 382. Looped element 304 is in the first configuration, thereby facilitating encompassing of epiglottis 226 by loop 352 as laryngoscope blade 302 is inserted into vallecula 224. An elongated member is then introduced into oral cavity 208 and distally advanced until a tip thereof passes through loop 352, reaching a position proximal to corniculate tubercle 252, essentially as described in the description of FIGS. 2A-2B. When the elongated member is thin, e.g. when the elongated member is a bougie introducer, looped element 304 is then switched to the second configuration (i.e. elongated wire 350 is proximally pulled, thereby contracting arched portion 332). When the elongated member is large caliber, e.g. when the elongated member is an (large caliber) endotracheal tube, instead of being switched to the first configuration, looped element 304 is switched to one of the mid-configurations; the second configuration being too narrow to accommodate the endotracheal tube or easy advancement of the endotracheal tube through the second arch (defined by the second configuration). As the elongated member is distally advanced farther, loop 352 directs the elongated member into laryngeal inlet 262, essentially as described in the description of FIG. 2B.

During laryngoscope blade 302 insertion into vallecula 224, extension angle β can be adjusted, e.g. by distally pushing or proximally pulling flexible rod 340. Such adjustment of extension angle β may ease encompassing of epiglottis 226 by loop 352. Extension angle β may also be adjusted after looped element 304 has been switched from the first configuration to the second configuration or to one of the mid-configurations. Such adjustment of extension angle β may help in guiding the elongated member through loop 352.

In embodiments wherein elongated wire 350 is rigid, looped element 304 can also be switched back and forth between the mid-configurations and the second configuration. For example, if after switching to the second configuration it is found that the second configuration does not allow for an easy (distal) advancement of the elongated member, looped element 304 may be loosened to a mid-configuration defining a larger arch, and, if need be, subsequently slightly tightened (i.e. switched to a more contracted mid-configuration). Or, for example, switching from the first configuration to the second configuration may be impossible—the elongated member being too thick to allow attaining the second configuration—so that looped element 304 is switched instead to one of the mid-configurations. Looped element 304 may then have to be loosened to allow for an easy advancement of the elongated member.

In some embodiments, loop 352 includes a loop top portion 396 which is made of heavier and less flexible material than a rest of loop 352, and which is little curved. In some embodiments, loop top portion 396 is substantially straight. Loop top portion 396 includes loop top 368, in the middle thereof. In some embodiments, loop top portion 396 has a length of about 2.5 cm, of about 3 cm, or even of about 4 cm (and may be wider than blade distal portion 314, which may have a width of about 2 cm: a Macintosh 4 blade, for example has a width of 1.8 cm), while in the second configuration arched portion 332 has a length of about 4 cm, of about 5 cm, or even of about 6 cm (in the first configuration arched portion 332 has a length of about 9 cm, 10 cm, or even of about 11 cm). When looped element 304 is switched from the first configuration to the second configuration or to one of the mid-configurations, due to loop top portion 396 resilience, loop top portion 396 retains the shape thereof. Consequently, the second arch, or an arch defined by one of the mid-configurations, e.g. arch 332, is wider than blade distal portion 314, which may ease insertion of large caliber instruments during ETI. Further, loop top portion 396 may be covered in Teflon™, further easing the insertion of large caliber instruments during ETI by reducing friction between the instruments and loop 352.

FIG. 4 schematically depicts a laryngoscope 400, including a laryngoscope blade 402 and a guiding assembly 406, according to some embodiments of the present disclosure. Guiding assembly 406 is detachably mountable on laryngoscope blade 402, as elaborated on below. Laryngoscope blade 402 is elongated and includes a blade proximal portion 412 and a blade distal portion 414. Laryngoscope blade 402 further includes a blade superior surface 416, a blade inferior surface 418, and opposite lateral edges: a blade first edge 422 and a blade second edge 424. A blade tip 426 is defined by the distal edge of blade distal portion 414.

A length of laryngoscope blade 402 is indicated by a line L5, which extends from the proximal edge (not numbered) of blade proximal portion 412 to blade tip 426, midway between blade edges 422 and 424. Laryngoscope blade 402 is essentially similar to laryngoscope blade 102 and further includes an elongated housing 428, mounted perpendicularly to laryngoscope blade 402 length at the proximal edge of blade proximal portion 402. Elongated housing 428 extends superiorly from a housing inferior end 1446 to a housing superior end 1448. Housing superior end 1448 includes a housing slot 430, extending transversely to line L5, and which is level with blade superior surface 416.

Guiding assembly 406 includes a hand grip 432, a tubular member 434, which is elongated, and a looped element 436. Hand grip 432 includes a handle 442 and a mounting bar 444. Handle 442 includes a handle elongate portion 446 and a handle superior portion 448 positioned superiorly relative to handle elongate portion 446. Handle elongate portion 446 and handle superior portion 448 are joined at a handle joint 452 (shown in FIG. 4B), with handle superior portion 448 projecting at an angle (not indicated) relative to handle elongate portion 446, as elaborated on below. Mounting bar 444 includes a mounting bar superior portion 454 and a mounting bar inferior portion 456. Mounting bar 444 is configured to be slid into elongated housing 428 (via housing slot 430), as elaborated on below.

Handle 442 and mounting bar 444 are mechanically connected at handle joint 452 and mounting bar superior portion 454, respectively, through connecting bars 458. Handle elongate portion 446 and mounting bar 444 subtend a pivot angle γ. Handle 442 is configured for a pivoting motion about handle joint 452, that is to say, a motion which alters pivot angle γ, as elaborated on below.

Tubular member 434 extends from a member proximal portion 462 to a member distal portion 464. Tubular member 434 is mounted on mounting bar 444 at member proximal portion 462 and a superior tip (not numbered) of mounting bar superior portion 454, respectively. In some embodiments member proximal portion 462 is mounted substantially at right angles to mounting bar 444.

A spring 466 is longitudinally disposed within tubular member 434 at member proximal portion 462. In some embodiments, spring 466 is a spring coil. In some embodiments, spring 466 is connected at a proximal end thereof (not numbered) to a member proximal tip 468 (that is to say, the proximal edge of member proximal portion 462). In some embodiments, member proximal tip 468 is walled, except for a hole (not shown) for admitting a wire therethrough, thereby blocking spring 466 at the proximal end thereof.

A connector 470 is attached (e.g. glued) to member distal portion 464 at an inferior portion thereof (not numbered). In some embodiments, tubular member 434 and connector 470 may be integrally formed. Connector 470 is configured to be detachably mounted on blade distal portion 414, as elaborated on below. Connector 470 includes a connector body 472. Connector body 472 is elongated and positioned transversely relative to the length of tubular member 434, transversely extending from a connector first edge 474 to a connector second edge 476. Connector body 472 further includes a superior surface (not numbered). A connector first leg 482 and a connector second leg 484 project in the inferior direction from an inferior surface (not numbered) of connector body 472. Connector legs 482 and 484 are configured for snap-in engagement with blade distal portion 414, as elaborated on below.

A first spring coil 486 and a second spring coil 488 are mounted on the superior surface of connector body 472. First spring coil 486 is mounted proximately to connector first edge 474 and second spring coil 488 is mounted proximately to connector second edge 476. First spring coil 486 is mounted such as to extend superiorly relative to connector body 472 and transversely relative thereto (that is to say, side-ways and away from connector body 472). Similarly, second spring coil 488 is mounted such as to extend superiorly relative to connector body 472 and transversely relative thereto (that is to say, side-ways and away from connector body 472). A functionality of spring coils 486 and 488 is elaborated on below.

Looped element 436 includes an elongated wire 490 and a loop 492. Elongated wire 490 extends from a wire proximal end 494 to a wire distal end 496, and includes a wire proximal portion 1402 and a wire distal portion 1404. A disc 1406 is disposed within member proximal portion 462, perpendicularly to the length of tubular member 434 and adjacently to the distal end (not numbered) of spring 466. Wire proximal portion 1402 includes wire proximal end 494 and is connected on the second end (not numbered) thereof to disc 1406. Wire distal portion 1404 includes wire distal end 496 and is connected on the second end (not numbered) thereof to disc 1406. Wire proximal portion 1402 is mechanically associated with handle 442, as elaborated on below.

Loop 492 includes a loop first arm 1412 and a loop second arm 1414, each extending from a loop base 1416 to a loop top 1418. Loop first arm 1412 and loop second arm 1414 are joined at loop top 1418. Elongated wire 490 and loop 492 are joined at wire distal end 496 and at loop base 1416, respectively. Loop first arm 1412 and loop second arm 1414 pass through first spring coil 486 and second spring coil 488, respectively.

FIG. 4B schematically depicts a back-view of hand grip 432. Handle elongate portion 446 includes a projecting member 1422. Projecting member 1422 is shaped substantially as a nail and proximally projects from handle elongate portion 446. Handle superior portion 448 includes a first notch 1426 and a second notch 1428 on a handle superior portion edge 1432 (that is to say, the superior end of handle superior portion 448). A first ring 1436 and a second ring 1438 are mounted on handle joint 452, inferiorly to first notch 1426 and to second notch 1428, respectively. First ring 1436 mounting is such that a symmetry axis thereof (not shown) points in the superior direction. Similarly, second ring 1438 mounting is such that a symmetry axis thereof (not shown) points in the superior direction.

Wire proximal end 494 is detachably connected to one of connecting bars 458. For example, one of connecting bars 458 may include a hook 1442 and wire proximal end 494 may include a slip-on ring 1444, configured to be slipped on hook 1442. Proximal wire portion 1402 includes three wire segments: a wire first segment 1452, a wire second segment 1454, and a wire third segment 1456. Wire first segment 1452 extends from hook 1442, passing through second notch 1428, into second ring 1438. Wire second segment 1454 extends from second ring 1438 to projecting member 1422, winds half-way about projecting member 1422, and extends therefrom into first ring 1436. Wire third segment 1456 extends from first ring 1436, via first notch 1426, into member proximal portion 462 (in embodiments wherein member proximal tip 468 is walled, through the hole in the wall) and thereby onto disc 1406.

Rings 1436 and 1438 function to ensure that along handle 442 wire proximal portion 1402 extends in parallel to handle 442, thereby substantially fixing a length of wire proximal portion 1402 which extends from first notch 1426 to second notch 1428, half-winding along the way around projecting member 1422. In some embodiments, handle 442 does not include rings 1436 and 1438, and projecting member 1422 is not mounted on handle elongate portion 446, being mounted instead on handle superior portion 448.

In some embodiments, blade distal portion 414 includes a first slot 1462 and a second slot 1464. Connector first leg 482 and connector second leg 484 include a first rib 1466 and a second rib 1468, respectively. First rib 1466 forms a protrusion on connector first leg 482, pointing laterally. Similarly, second rib 1468 forms a protrusion on connector second leg 484, pointing laterally. First rib 1466 and second rib 1468 are configured to facilitate snap-in engagement between connector first leg 482 and first slot 1462 and between connector second leg 484 and second slot 1464. In some embodiments, blade distal portion 414 includes a pair of opposite notches (not shown) on blade first edge 420 and blade second edge 422, respectively. In such embodiments, first rib 1466 points medially and second rib 1468 points medially, thereby facilitating snap-in engagement between connector 470 and blade distal portion 414.

Guiding assembly 406 can be mounted on laryngoscope blade 402 by sliding mounting bar 444 into elongated housing 428, via housing slot 430, and inserting connector first leg 482 and connector second leg 484 into first slot 1462 and second slot 1464, respectively. When guiding assembly 406 is mounted on laryngoscope blade 402, tubular member 434 is longitudinally disposed along blade superior surface 416. In some embodiments, tubular member 434 is flexible and thereby configured to be mountable on different embodiments of laryngoscope blade 402, differing from one another in the shape of blade superior surface 416. In some embodiments laryngoscope blade 402 and guiding assembly 406 may be integrally formed, or, for example, tubular member 434 may be glued onto blade superior surface 416. In some embodiments, wherein guiding assembly 406 includes a ratchet mechanism, as elaborated on below, when guiding assembly 406 is mounted on laryngoscope blade 402, member proximal portion 462 may be superiorly elevated relative to blade superior surface 416 at blade proximal portion 412 in order to accommodate the ratchet mechanism. Loop 492 includes an arched portion 1472. Arched portion 1472 is similar to expanded arch 132a, being substantially shaped as a horseshoe and extending superiorly relative to blade superior surface 416 at blade distal portion 414 (when guiding assembly 406 is mounted on laryngoscope blade 402). Arched portion 1472 includes an arched portion first arm 1474 and an arched portion second arm 1476. Arched portion first arm 1474 consists of a portion of loop first arm 1412, which extends from the inferior end of first spring coil 486 to loop top 1418. Arched portion second arm 1476 consists of a portion of loop second arm 1414, which extends from the inferior end of second spring coil 488 to loop top 1418.

Similarly to looped element 104, looped element 436 is switchable from a first configuration to a second configuration. In the first configuration, arched portion 1472 defines an expanded arch (shown in FIG. 4A wherein arched portion 1472 is depicted as fully expanded) similar to expanded arch 132a. In the second configuration, arched portion 1472 defines a contracted arch (not shown) similar to contracted arch 132b. Further, looped element 436 is continuously switchable from the first configuration to the second configuration, defining a range of mid-configurations with increasingly smaller arches, and from the second configuration to the first configuration, as elaborated on below. In the first configuration, spring 466 is relaxed, while in the second configuration spring 466 is compressed.

Spring coils 486 and 488 help sustain the superior extension of the arches from blade distal portion 414 (when guiding assembly 406 is mounted on laryngoscope blade 402). In particular, spring coils 486 and 488 help maintain arched portion 1472 superior and lateral extension relative to blade distal portion 414 by supporting and directing arched portion first arm 1474 and arched portion second arm 1476, respectively (which pass through spring coils 486 and 488, respectively). It is noted that spring coils 486 and 488 are flexible, thereby facilitating safely accommodating different epiglottic and laryngopharyngeal architectures, which may greatly vary from one subject to another. In some embodiments, the spring coils can be coated by a non-stick material such as Teflon™. In some embodiments, the spring coils can be covered by a PTFE coated sleeve.

The pivoting motion allows switching between a first handle configuration, shown in FIG. 4A, and a second handle configuration (not shown). In the second handle configuration pivot angle γ is smaller than in the first handle configuration. For example, in the first configuration pivot angle γ may be about 30° and in the second configuration pivot angle γ may be about 10°. Further, in the second handle configuration, handle superior portion edge 1432 is positioned farther from member proximal tip 468 than in the first handle configuration, being substantially proximally shifted, or proximally and inferiorly shifted, relative to a position thereof in the first handle configuration. In some embodiments, in the first handle configuration, handle superior portion 448 is substantially parallel to mounting bar 444.

In the first handle configuration, looped element 436 is in the first configuration. In the second handle configuration, looped element 436 is in the second configuration.

To switch from the first handle configuration to the second handle configuration, elongated portion 446 is fully pressed towards mounting bar 444 and pivot angle γ is decreased. The resultant proximal pulling of handle superior portion edge 1432 results in an increase in the length of wire first segment 1452, since elongated wire 490 is non-stretchable, in wire third segment 1456 being proximally pulled to compensate for the increase in wire first segment 1452 length (wire second segment 1454 length remains substantially unchanged). Wire third segment 1456 proximally pulls disc 1406, which in turn compresses spring 466 by pushing spring 466 in the proximal direction. Further, disc 1406 proximally pulls distal wire portion 1404, which in turn proximally pulls loop base 1416 into tubular member 434. Consequently, loop base 1416 proximally pulls loop first arm 1412 and loop second arm 1414, thereby shortening arched portion first arm 1474 and arched portion second arm 1476, respectively. Arched portion first arm 1474 and arched portion second arm 1476 shortening results in the shrinking (i.e. contraction) of arched portion 1472, thereby bringing looped element 436 into the second configuration (or into one of the mid-configurations if handle elongate portion 446 is not fully pressed).

In operation, laryngoscope 400 is inserted into oral cavity 208, such that blade distal portion 414 is inserted into vallecula 224, with arched portion 1472 encompassing epiglottis 226. An elongated member, such as bougie introducer 206, is next introduced into oral cavity 208 and distally advanced until a distal tip of the elongated member has passed through arched portion 1472 and is positioned proximally relative to corniculate tubercle 252, essentially as described in the description of FIG. 2A. Looped element 436 is then switched from the first configuration to the second configuration, or to one of the mid-configurations, and arched portion 1472 is contracted, as described above. The elongated member is then distally advanced farther, being directed by the contracted arch (or by an intermediate arch defined by one of the mid-configurations when handle elongate portion 446 is not fully pressed) into laryngeal inlet 262, essentially as described above in the description of FIG. 2B.

In some embodiments, first slot 1462 and second slot 1464 are elongated, extending along the length of blade distal portion 414, such that when mounted thereon, connector first leg 482 and connector second leg 484 occupy only a portion along the respective lengths of first slot 1462 and second slot 1464. For example, each of connector legs 482 and 484 may occupy a half, a third, a fifth, or even a tenth of the lengths of slots 1462 and 1464, respectively. Further, slots 1462 and 1464 are configured to allow for connector 470 to be shifted along blade superior surface 416 when connector 470 is snap-engaged thereto, as elaborated on below.

A toothed wheel 1482 is mounted on mounting bar superior portion 454 between, and in parallel to, connecting bars 458. Member proximal portion 462 includes teeth 1484 evenly disposed along the length of tubular member 434 on an inferior portion thereof (not numbered). Blade proximal portion 412 includes a blade slit 1486 extending from housing slot 430 in the distal direction. Elongated housing 428 includes a pair of housing slits: a housing first slit 1488 and a housing second slit 1489. Housing slits 1488 and 1489 extend in the inferior direction on a housing proximal wall 1490 and on a housing distal wall 1492, respectively, from housing superior end 1448. A length of blade slit 1486 may be between about 1 cm to about 2 cm. Lengths of housing slits 1488 and 1489 may be between about 1 cm to about 3 cm.

Slits 1486, 1488, and 1489 are configured to accommodate toothed wheel 1482 and teeth 1484 when guiding assembly 406 is mounted on laryngoscope blade 402. Teeth 1484 are disposed superiorly to blade slit 1486 when guiding assembly 406 is mounted on laryngoscope blade 402. Teeth 1484 and toothed wheel 1482 are configured for a ratchet-like engagement, thereby facilitating pulling tubular member 434 in the proximal direction (while maintaining hand grip 432 and laryngoscope blade 402 stationary). In some embodiments, the ratchet-like engagement is reversible, that is to say, two-way, thereby additionally facilitating pushing tubular member 434 in the distal direction. Toothed wheel 1482 may be turned, using, for example, a lever (not shown) mounted thereon.

An arched portion base 1405 is defined by the inferior end of arched portion first arm 1474 and the inferior end of arched portion second arm 1476. A mounting site 1415 is defined by a segment of blade first edge 422 adjacent to first slot 1464 and by a segment of blade second edge 424 adjacent to second slot 1466. A mounting mechanism is provided by first spring coil 486 and second spring coil 488. The mounting mechanism (together with elongated wire 490 and hook 1442) substantially restricts arched portion base 1405 to mounting site 1415 when connector 470 is snap-engaged to slots 1464 and 1466.

In embodiments wherein laryngoscope 400 includes wheel 1482 and teeth 1484, and slots 1464 and 1466 are elongated, mounting site 1415 is defined by a segment of blade first edge 422, adjacent to the distal end of first slot 1464, and by a segment of blade second edge 424, adjacent to the distal end of second slot 1466. A second mounting site 1435 is defined by a segment of blade first edge 422, adjacent to the proximal end of first slot 1464, and by a segment of blade second edge 424, adjacent to the proximal end of second slot 1466. By turning toothed wheel 1482, and thereby proximally shifting tubular member 434, arched portion base 1405 can be controllably shifted from mounting site 1415 to second mounting site 1435 (and vice-versa by turning toothed wheel 1484 in the opposite sense), and in some embodiments to additional mounting sites (not numbered) there between.

In some embodiments, hand grip 432 further includes a fourth spring 1494. Fourth spring 1494 is mounted between mounting bar 444 and handle elongate portion 446, being attached on one end thereof to mounting bar 444 and on the second end thereof to handle elongate portion 446. Fourth spring 1494 acts to provide additional resistance (beyond that provided by spring 466) to pressing handle elongate portion 446, thereby in some embodiments providing increased control in switching looped element 436 from the first configuration to the mid-configurations. In embodiments including fourth spring 1494, housing slit 1488 length may be about 5 cm, about 8 cm, or even about 10 cm or longer, in order to accommodate fourth spring 1494 (as well as toothed wheel 1482). In some embodiments, housing slit 1488 may extend from housing superior end 1448 to housing inferior end 1446.

According to some embodiments, tubular member 434 can be magnetically adhered to blade superior 416. For example, tubular member 434 may have magnets (not shown) disposed on the inferior surface thereof. In such embodiments, elongated wire 490 and loop 492 may be made of plastic.

According to some embodiments, tubular member 434 is mounted on, and disposed along, blade inferior surface 418, with loop 492 encompassing blade distal portion 414. In such embodiments, first spring coil 486 and second spring coil 488 may be mounted on blade first edge 422 and blade second edge 424, respectively.

Making reference again to FIG. 3, in some embodiments laryngoscopy device 300 includes a pair of spring coils, such as spring coils 486 and 488, mounted on blade superior surface 316 at blade distal portion 314 similarly to the mounting of spring coils 486 and 488 on connector 470.

FIG. 5 schematically depicts a laryngoscopy device 500, including a laryngoscope blade 502 and a looped element 504, according to some embodiments of the present disclosure. Laryngoscopy device 500 provides a specific embodiment of laryngoscopy device 100. Laryngoscope blade 502 is elongated and includes a blade proximal portion 512 and a blade distal portion 514 (per the directions defined in FIG. 1 which also apply for FIG. 5). Laryngoscope blade 502 further includes a blade superior surface 516, a blade inferior surface 518, and opposite lateral edges: a blade first edge 520, and a blade second edge 522. A blade tip 524 is defined by the distal edge of blade distal portion 514. According to some embodiments, laryngoscope blade 502 may be mounted on a handle, such as handle 210.

A length of laryngoscope blade 502 is indicated by a line L6, which extends from the proximal edge (not numbered) of blade proximal portion 512 to blade tip 524, midway in between blade edges 520 and 522.

Blade distal portion 514 includes a pair of parallel rails: a first rail 532 and a second rail 534. Each of first rail 532 and second rail 534 is shaped as a narrow and elongated bar. First rail 532 proximally extends from blade tip 524 along blade distal portion 514, in parallel to and proximately to blade first edge 520. First rail 532 is elevated relative to blade superior surface 516, e.g. by about 0.3 cm, and is joined thereto by first rail legs 536. First rail legs 536 are positioned at the two ends (not numbered) of first rail 532, projecting in the superior direction relative to blade superior surface 516. Similarly, second rail 534 proximally extends from blade tip 524 along blade distal portion 514, in parallel to and proximately to blade second edge 522. Second rail 534 is elevated relative to blade superior surface 516, e.g. by about 0.3 cm, and is joined thereto by second rail legs 538. Second rail legs 538 are positioned at the two ends (not numbered) of second rail 534, projecting in the superior direction relative to blade superior surface 516. In some embodiments, first rail 532 and second rail 534 are between about 2 cm and about 6 cm long, or between about 3 cm to about 5 cm long. First rail 532 and second rail 534 functionality is elaborated on below.

Blade tip 524 includes a canal 526, in the form of an elongated passage, extending between two opposite openings: a canal first opening 528, at blade first edge 520, and a canal second opening 530 at blade second edge 522. Blade tip 524 further includes a bore (not shown), bisecting canal 526 and pointing in the distal direction. A first push-button 542 is located on blade tip 524, extending into blade tip 524 bore. A spring (not shown) is positioned within canal 526. Canal 526 spring is mechanically connected to first push-button 542, such that when canal 526 spring is relaxed, first push-button 542 projects outside of blade tip 524 in the distal direction (in addition to proximally extending into blade tip 524 bore). When first push-button 542 is pressed, first push-button 542 is proximally pushed into blade tip 524 bore and canal 526 spring is compressed. When the pressing is ceased, canal 526 spring pushes back first push-button 542 (i.e. in the distal direction).

First push-button 542 includes a hole (not shown). When first push-button 542 is pressed, first-push button 542 hole is aligned with canal 526. When first push-button 542 is not pressed, first-push button 542 hole is only partially aligned with canal 526. First push-button 542 functionality is further elaborated on below.

A second push-button 544 is located on blade inferior surface 518 at blade distal portion 514. A stopper 546 is located oppositely to second push-button 544 on blade superior surface 516. Stopper 546 includes a frame mount 552 and a stopper frame 554 mounted thereon (i.e. stopper frame 554 is elevated relative to blade superior surface 516). In some embodiments, stopper frame 554 is shaped substantially as a square bracket (with rounded corners) and is mounted transversely relative to laryngoscope blade 502 length (i.e. transversely to line L6). In some embodiments, stopper frame 554 includes two pairs of frame legs: a first pair of legs 556 and a second pair of legs 558, which are positioned proximately to blade first edge 520 and to blade second edge 522, respectively, and which project in the inferior direction from stopper frame 554 towards blade superior surface 516. The two legs of first pair of legs 556 are positioned on the two sides of first rail 532. Similarly, the two legs of second pair of legs 558 are positioned on the two sides of second rail 534.

Stopper 546 is mechanically associated with second push-button 544, such that when second push-button 544 is pressed (i.e. pushed superiorly), stopper 546 is pushed in the superior direction. For example, in some embodiments second-push button 544 includes a spring (not shown) mounted perpendicularly to blade inferior surface 518. Blade distal portion 514 includes a bore (not shown). Frame mount 552 extends through blade distal portion 514 bore and the inferior end (not shown) of frame-mount 552 is attached to second push-button 544 spring. In some embodiments second push-button 544 does not include a spring, but is made of an elastic material, such that when pressed, second push-button 544 superiorly pushes frame mount 552 (which extends through blade distal portion 514 bore and which is attached to second push-button 544). Second push-button 544 and stopper 546 functionalities are further elaborated on below.

A first spring 562 and a second spring 564 are mounted on first rail 532 and second rail 534, respectively. In some embodiments, both first spring 562 and second spring 564 are coil springs. First spring 562 longitudinally extends from a first spring distal end 572 to a first spring proximal end 574. First spring distal end 572 is fixed proximately to blade tip 524. For example, first spring distal end 572 may be attached (e.g. glued or welded) to the distal leg of the two legs constituting first rail legs 536.

Similarly, second spring 564 longitudinally extends from a second spring distal end 576 to a second spring proximal end 578. Second spring distal end 576 is fixed proximately to blade tip 524. First spring 562 has a relaxation length (i.e. a length thereof when not compressed or stretched) longer than first rail 532, e.g. 9 cm when first rail 532 is 6 cm long. Similarly, second spring 564 has a relaxation length longer than second rail 534.

In FIG. 5 first spring 562 and second spring 564 are shown proximally extending until first pair of legs 556 and second pair of legs 558, respectively. Both first spring 562 and second spring 564 are compressed (e.g. measuring 3 cm long). First pair of legs 556 and second pair of legs 558 bar first spring proximal end 574 and second spring proximal end 578 from proximally advancing along first rail 532 and second rail 534, respectively. When second-push button 544 is pressed, stopper frame 554 and first pair of legs 556 and second pair of legs 558 are lifted, that is to say superiorly pushed away from blade superior surface 516. Consequently, first spring proximal end 574 and second spring proximal end 578 are no longer barred by first pair of legs 556 and second pair of legs 558, respectively, and move proximally along first rail 532 and second rail 534, until barred from further proximal advance by the proximal leg of first rail legs 536 and the proximal leg of second rail legs 538, respectively. First spring 562 and second spring 564 functionality is further elaborated on below.

Looped element 504 includes an elongated wire 580. Elongated wire 580 extends from a wire first end 582 to a wire second end 584, and includes a wire first portion 588, a wire second portion 590, and a wire arched portion 592. Wire first portion 588 includes wire first end 582. Wire second portion 590 includes wire second end 584.

Wire first end 582 and wire second end 584 are attached to first spring proximal end 574 and second spring proximal end 578, respectively. Wire first portion 588 is defined as a portion of elongated wire 580, which extends from first spring proximal end 574 to canal second opening 530. Wire first portion 588 extends outside of first spring 562, and passes through canal 526 (entering canal 526 via canal first opening 528 and passing through first push-button 542 hole). Wire second portion 590 is defined as a portion of elongated wire 580, which extends from second spring proximal end 578 to canal first opening 528. Wire second portion 590 extends outside of second spring 564, and passes through canal 526 (entering canal 526 via canal second opening 530 and passing through first push-button 542 hole). In some embodiments, wire first portion 588 and wire second portion 590 pass through first spring 562 and second spring 564, respectively. Wire first portion 588 is joined to wire arched portion 592 at canal second opening 530. Wire second portion 590 is joined to wire arched portion 592 on canal first opening 528. When first-push button 542 is not pressed, first push-button 542 hole is only partially aligned with canal 526, thereby clamping (i.e. pressing) wire first portion 588 and wire second portion 590 between walls (not shown) of first push-button 542 hole and walls (not shown) of canal 526. The clamping of wire first portion 588 and wire second portion 590 prevents any movement of wire first portion 588 and wire second portion 590 along canal 526 (similarly to a drawstring mechanism).

Wire arched portion 592 is pre-shaped, similarly to a horseshoe, but in some embodiments is additionally bent as elaborated on below. Wire arched portion 592 is made of a material(s) which is both rigid and flexible, similarly to a catheter guidewire, as elaborated on above in the descriptions of looped element 104 and looped element 304. Wire arched portion 592 rigidity is sufficient to maintain the (pre-shaped) shape thereof, unless acted on with sufficiently large forces. Wire arched portion 592 flexibility ensures that wire arched portion 592 returns to the (pre-shaped) shape thereof, after having been made to change shape (e.g. bent), so long as the applied forces were not too large. In particular, wire arched portion 592 rigidity ensures that wire arched portion 592 maintains the (pre-shaped) shape thereof when looped element 504 is mounted on laryngoscope blade 502, while wire arched portion 592 flexibility ensures that wire arched portion 592 changes shape when pressed against walls of oral cavity 208 or walls of laryngopharynx 234, e.g. laryngopharyngeal posterior wall 232, during ETI.

Wire arched portion 592 includes an arched portion first arm 1502 and an arched portion second arm 1504. Arched portion first arm 1502 and arched portion second arm 1504 are joined at an arched portion top 1510. Similarly to looped element 104, looped element 504 is switchable from a first configuration, defining an expanded arch similar to expanded arch 132a, to a second configuration, defining a contracted arch (not shown) similar to contracted arch 132b. FIG. 5 depicts the first configuration, that is to say, wire arched portion 592 is shown expanded. Arched portion top 1510 is positioned superiorly relative to blade superior surface 516. Further, arched portion top 1510 is positioned proximally relative to blade tip 524. In some embodiments, in the first configuration, arched portion top 1510 may be positioned distally relative to blade tip 524, or neither distally nor proximally.

Arched portion first arm 1502 extends from canal second opening 530—whereat arched portion first arm 1502 is joined to wire first portion 588—to arched portion top 1510. Similarly, arched portion second arm 1504 extends from canal first opening 528—whereat arched portion second arm 1504 is joined to wire second portion 590—to arched portion top 1510. Arched portion first arm 1502 includes a first arm mid-point 1512 dividing arched portion first arm 1502 into two portions (not numbered), which in some embodiments may be substantially equally long. Arched portion second arm 1504 includes a second arm mid-point 1514 dividing arched portion second arm 1504 into two portions (not numbered), which in some embodiments may be substantially equally long. First arm mid-point 1512 is positioned laterally and superiorly relative to canal second opening 530. Second arm mid-point 1514 is positioned laterally and superiorly relative to canal first opening 528. In some embodiments, in the first configuration, first arm mid-point 1512 and second arm mid-point 1514 are positioned distally relative to blade tip 524, while arched portion top 1510 is positioned proximally relative to blade tip 524. That is to say, in some embodiments, in the first configuration, wire arched portion 592 is proximally curved/bent.

An arched portion base 1526 is defined by the inferior end of arched portion first arm 1502 (which is joined to wire first portion 588) and the inferior end of arched portion second arm 1504 (which is joined to wire second portion 590). A mounting site 1530 is defined by canal first opening 528 and canal second opening 530. A mounting mechanism is provided by canal 526 walls and first push-button 542. The mounting mechanism (together with first spring 562 and second spring 564) restricts arched portion base 1526 to mounting site 1530, that is to say, the inferior end of arched portion first arm 1502 to canal second opening 530 and the inferior end of arched portion second arm 1504 to canal first opening 528.

In operation, laryngoscope device 500 is inserted into oral cavity 208, such that blade distal portion 514 is inserted into vallecula 224, with wire arched portion 592 encompassing epiglottis 226. An elongated member, such as bougie introducer 206, is next introduced into oral cavity 208 and distally advanced until a distal tip of the elongated member has passed through wire arched portion 592 and is positioned proximally relative to corniculate tubercle 252, essentially as described in the description of FIG. 2A. Looped element 504 is then switched to the second configuration, thereby directing the elongated member into laryngeal inlet 262, essentially as described in the description of FIG. 2B.

To switch from the first configuration, depicted in FIG. 5, to the second configuration, laryngoscope blade 502 is pushed in both the distal direction (against the distalmost point of vallecula 224) and the anterior direction (against base of the tongue 238). The distal pushing of laryngoscope blade 502 causes first push-button 542 to be pressed, bringing first push-button 542 hole into alignment with canal 526, with the result that wire first portion 588 and wire second portion 590 are no longer clamped between canal 526 walls and the walls of first push-button 542 hole walls. The anterior pushing of laryngoscope blade 502 causes second push-button 544 to be pressed. Consequently, stopper frame 554 is pushed in the posterior direction, releasing first spring 562 and second spring 564, that is to say, first spring proximal end 574 and second spring proximal end 578 are no longer barred by first pair of legs 556 and second pair of legs 558, respectively. Since wire first end 582 and wire second end 584 are attached to first spring proximal end 574 and second spring proximal end 578 (and since wire first portion 588 and wire second portion 590 are no longer clamped inside canal 526), the release of first spring 562 and second spring 564 releases also wire first portion 588 and wire second portion 590.

Since first spring 562 is compressed, particularly in the first configuration, and since the relaxation length of first spring 562 is longer than first rail 532, first spring 562 partially decompresses until first spring proximal end 574 is barred from further proximal advance by the proximal leg of first rail legs 536. Similarly, since second spring 564 is compressed, particularly in the first configuration, and since the relaxation length of second spring 564 is longer than second rail 534, second spring 564 partially decompresses until second spring proximal end 578 is barred from further proximal advance by the proximal leg of second rail legs 538.

Since wire first end 582 is attached to first spring proximal end 574 (and since first-push button 542 is pressed and wire first portion 588 is consequently not clamped), first spring 562 decompression results in wire first end 582 being (proximally) advanced together with first spring proximal end 574. Similarly, since wire second end 584 is attached to second spring proximal end 578 (and since first-push button 542 is pressed and wire second portion 590 is consequently not clamped), second spring 564 decompression results in wire second end 584 being (proximally) advanced together with second spring proximal end 578. Consequently, each of wire first portion 588 and wire second portion 590 is lengthened, and wire arched portion 592 shrinks (i.e. arched portion first arm 1502 and arched portion second arm 1504 are shortened), thereby bringing looped element 504 into the second configuration.

In some embodiments, due to looped element 504 pre-shaping, in the second configuration, arched portion top 1510 is not proximally positioned relative to blade tip 524, and may even be positioned distally relative thereto. Such pre-shaping of looped element 504 may facilitate insertion of bougie introducer 206 during ETI.

In some embodiments, laryngoscopy device 500 includes a flexible rod, such as flexible rod 340. The flexible rod is connected at a distal tip thereof, such as rod tip 382, to looped element 504, e.g. at arched portion top 1510. By distally pushing and proximally pulling the flexible rod, wire arched portion 592 shape and an orientation thereof may be adjusted, essentially as described in the description of FIG. 3. Further, the flexible rod may be used to help switch from the second configuration back to the first configuration. To do so, both first push-button 542 and second push-button 544 are pressed, to allow wire first end 582 and wire second end 584 to be distally pulled. By proximally pulling the flexible rod, arched portion top 1510 is proximally pulled, thereby expanding wire arched portion 592 and bringing looped element 504 into the first configuration. In particular, arched portion first arm 1502 and arched portion second arm 1504 are pulled with the result that first spring proximal end 574 and second spring proximal end 578 are proximally pulled and first spring 562 and second spring 564 are compressed. First push-button 542 and second push-button 544 are then released. Consequently, wire first portion 588 and wire second portion 590 become clamped in canal 526, and, further, first pair of legs 556 and second pair of legs 558 are pushed towards blade superior surface 516, thereby barring first spring proximal end 574 and second spring proximal end 578, respectively (and securing looped element 504 in the first configuration).

According to some embodiments, a third arm 1532 and a fourth arm 1534 extend from, or from about, first arm mid-point 1512 and second arm mid-point 1514, respectively. A second arched portion 1540 (marked by a dashed line) is formed by third arm 1532 and fourth arm 1534, which are joined at a second arched portion top 1542. Second arched portion top 1542 is positioned superiorly relative to blade superior surface 516 and proximally relative to arched portion top 1510. In some embodiments, second arched portion 1540 is connected to wire arched portion 592 by e.g. a thin metal net or plastic film (which may be collapsible or flexible). Second arched portion 1540 may help in guiding bougie introducer 206 (or other types of elongated intubation member, such an endotracheal tube) into laryngeal inlet 262 during ETI, by providing better support and the possibility of angulation.

It is noted that a guiding instrument, resembling laryngoscopy device 100, may be used for increasing control and accuracy in minimally invasive procedures. In some embodiments, the guiding instrument is longer than laryngoscopy device 100, for example, the guiding instrument can be about 20 cm long, about 30 cm long, or even about 50 cm long. In some embodiments, the guiding instrument may be mounted on (e.g. glued to) a laparoscopic or thoracoscopic trocar or an endoscope at the respective distal ends thereof, such that an arch (similar to expanded arch 132a) defined by a looped element (similar to looped element 104) of the guiding instrument projects away from the guiding instrument. In some embodiments, the guiding instrument can be flexible and attached to a flexible endoscope. To controllably maneuver a device (e.g. a grasping device, a surgical stapler, or a fiber-optic light source) proximately to the laparoscopic trocar or the endoscope, the laparoscopic trocar or the endoscope, with the guiding instrument mounted thereon, as described above, may be inserted into a hollow cavity (e.g. stomach, bowel, abdomen, urinary bladder, uterus, ear canal, nostril, etc.). Next, the device may be advanced from the site of insertion (of the laparoscopic trocar or the endoscope), or from a different site, until a distal tip of the device passes through the arch. The looped element is then switched from the first configuration to the second configuration. The device may have projecting elements that can be hooked to matching elements on the guiding instrument, enabling a secured connection there between.

According to an aspect of some embodiments, there is provided a laryngoscopy device (e.g. 100, 204, 300, 400, 500) including:
a laryngoscope blade (e.g. 102, 302, 402, 502), being elongated, having a blade distal portion (e.g. 114, 314, 414, 514), a blade first edge (e.g. 122, 322, 422, 520), and a blade second edge (e.g. 124, 324, 424, 522), opposite to the blade first edge; and
a looped element (e.g. 104, 304, 436, 504), switchable at least from a first configuration to a second configuration.

In the first configuration and in the second configuration the looped element defines an expanded arch (e.g. 132a, 1472, 592) and a contracted arch (e.g. 132b), respectively. The expanded arch has an expanded arch first arm (e.g. 134a, 1474) and an expanded arch second arm (e.g. 136a, 1476), joined at an expanded arch top (e.g. 138a, 1418, 1510). The contracted arch has a contracted arch first arm (e.g. 134b) and a contracted arch second arm (e.g. 136b), joined at a contracted arch top (e.g. 138b).

The expanded arch top is positioned superiorly relative to the blade distal portion. The expanded arch first arm at least partially extends laterally from about the blade first edge and the expanded arch second arm at least partially extends laterally from about the blade second edge.

According to some embodiments, the looped element (e.g. 104, 304, 436) is continuously switchable from the first configuration to the second configuration, across a range of mid-configurations, defining a respective range of increasingly smaller arches (e.g. 332), ranging from the expanded arch to the contracted arch.

According to some embodiments, the looped element (e.g. 104, 304, 436, 504) is additionally switchable from the second configuration to the first configuration.

According to some embodiments, the looped element (e.g. 104, 304, 436) is continuously switchable from the second configuration to the first configuration.

According to some embodiments, the contracted arch top (e.g. 138b) is positioned superiorly relative to the blade distal portion.

According to some embodiments, the blade distal portion is curved and the expanded arch (e.g. 132a, 1472, 592) and the contracted arch (e.g. 132b) are each configured such as (i) to encompass the epiglottis of a subject when the laryngoscopy device is inserted into the oral cavity of the subject with the blade distal portion being inserted into the vallecula of the subject, and (ii) to simultaneously facilitate guiding of an elongated intubation member through the expanded arch and through the contracted arch, respectively.

According to some embodiments, the elongated intubation member is a bougie introducer (e.g. 206), a stylet, or an endotracheal tube.

According to some embodiments, there is provided a laryngoscope including the laryngoscopy device and a handle (e.g. 210, 432), wherein the laryngoscope blade is mechanically connected to the handle.

According to an aspect of some embodiments, there is provided a laryngoscopy device (e.g. 100, 204, 400, 500) including:
a laryngoscope blade (e.g. 102, 402, 502), being elongated, having a blade distal portion (e.g. 114, 414, 514);
a looped element (e.g. 104, 436, 504), switchable at least from a first configuration to a second configuration; and
a mounting mechanism (e.g. 486 and 488, 526 and 542).

In the first configuration and in the second configuration the looped element respectively defines an expanded arch (e.g. 132a, 1472, 592), having an expanded arch base (e.g. 550a, 1405, 1526), and a contracted arch (e.g. 132b), having a contracted arch base (e.g. 550b).

The mounting mechanism supports (for example, mechanically supports) the expanded arch and the contracted arch in the first configuration and in the second configuration, respectively.

The mounting mechanism substantially restricts the expanded arch base and the contracted arch base to a mounting site (e.g. 170, 1415, 1530) on the blade distal portion.

According to some embodiments, an arch top (e.g. 138a, 1418, 1510) of the expanded arch is positioned superiorly relative to the blade distal portion.

According to some embodiments, the expanded arch base and the contracted arch base are controllably shiftable from the mounting site to at least one other mounting site (e.g. 1435) on the blade distal portion.

According to some embodiments, the expanded arch is wider than the blade distal portion.

According to an aspect of some embodiments, there is provided a method for performing laryngoscopy on a subject. The method includes the steps of:
providing any one of the laryngoscopy devices (e.g. 100, 204, 300, 400, 500) and an elongated intubation member (e.g. 206);
introducing the laryngoscopy device into the oral cavity of the subject with the looped element being in the first configuration;
maneuvering the laryngoscope blade such as to insert the blade distal portion into the vallecula of the subject, thereby encompassing the epiglottis of the subject with the expanded arch;
introducing the elongated intubation member into the oral cavity and distally advancing the elongated intubation member until a member distal tip thereof passes through the expanded arch and is proximally positioned relative to the corniculate tubercle of the subject;
switching from the first configuration to the second configuration thereby directing the member distal tip towards the laryngeal inlet of the subject; and
distally advancing the member distal tip into the laryngeal inlet of the subject.

According to some embodiments, the elongated intubation member is a bougie introducer (e.g. 206), and the method further includes, following the step of distally advancing the member distal tip into the laryngeal inlet of the subject, a step of mounting an endotracheal tube on the bougie introducer.

According to some embodiments, the elongated intubation member is an endotracheal tube.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A laryngoscopy device comprising:
a laryngoscope blade, being elongated, having a blade distal portion;
a looped element, switchable at least from a first configuration to a second configuration; and
a mounting mechanism;
wherein in the first configuration said looped element respectively defines an expanded arch, having an expanded arch base, and in the second configuration said looped element defines a contracted arch, having a contracted arch base;
wherein said mounting mechanism is configured to support said expanded arch and said contracted arch in the first configuration and in the second configuration, respectively; and
wherein said mounting mechanism is configured to restrict said expanded arch base and said contracted arch base to a mounting site on said blade distal portion.

2. The laryngoscopy device of claim 1, wherein said looped element is continuously switchable from the first configuration to the second configuration, across a range of mid-configurations, defining a respective range of increasingly smaller arches, ranging from said expanded arch to said contracted arch.

3. The laryngoscopy device of claim 1, wherein said looped element is additionally switchable from the second configuration to the first configuration.

4. The laryngoscopy device of claim 3, wherein said looped element is continuously switchable from the second configuration to the first configuration.

5. The laryngoscopy device of claim 1, wherein an arch top of said expanded arch is positioned superiorly relative to said blade distal portion.

6. The laryngoscopy device of claim 1, wherein said expanded arch is wider than said blade distal portion.

7. The laryngoscopy device of claim 5, wherein said expanded arch base and said contracted arch base are controllably shiftable from said mounting site to at least one other mounting site on said blade distal portion.

8. The laryngoscopy device of claim 1, wherein said blade distal portion is curved and said expanded arch and said contracted arch are each configured such as (i) to encompass the epiglottis of a subject when the laryngoscopy device is inserted into the oral cavity of a subject with said blade distal portion being inserted into the vallecula of the subject, and (ii) to simultaneously facilitate guiding of an elongated intubation member through said expanded arch and through said contracted arch, respectively.

9. The laryngoscopy device of claim 8, wherein said elongated intubation member is a bougie introducer, a stylet, or an endotracheal tube.

10. A laryngoscope comprising the laryngoscopy device of claim 1, and a handle, wherein said laryngoscope blade is connected to said handle.

11. A laryngoscopy device comprising:
a laryngoscope blade, being elongated, having a blade distal portion, a blade first edge, and a blade second edge, opposite to said blade first edge; and
a looped element, switchable at least from a first configuration to a second configuration;
wherein, in the first configuration and in the second configuration, said looped element defines an expanded arch and a contracted arch, respectively, wherein said expanded arch has an expanded arch first arm and an expanded arch second arm, joined at an expanded arch top, and said contracted arch has a contracted arch first arm and a contracted arch second arm, joined at a contracted arch top; and
wherein said expanded arch top is positioned superiorly relative to said blade distal portion, and said expanded arch first arm at least partially extends laterally from about said blade first edge and said expanded arch second arm at least partially extends laterally from about said blade second edge.

12. The laryngoscopy device of claim 11, wherein said looped element is continuously switchable from the first configuration to the second configuration, across a range of mid-configurations, defining a respective range of increasingly smaller arches, ranging from said expanded arch to said contracted arch.

13. The laryngoscopy device of claim 1, wherein said looped element is additionally switchable from the second configuration to the first configuration.

14. The laryngoscopy device of claim 13, wherein said looped element is continuously switchable from the second configuration to the first configuration.

15. The laryngoscopy device of claim 1, wherein said contracted arch top is positioned superiorly relative to said blade distal portion.

16. The laryngoscopy device of claim 1, wherein said blade distal portion is curved and said expanded arch and said contracted arch are each configured such as (i) to encompass the epiglottis of a subject when the laryngoscopy device is inserted into the oral cavity of a subject with said blade distal portion being inserted into the vallecula of the subject, and (ii) to simultaneously facilitate guiding of an elongated intubation member through said expanded arch and through said contracted arch, respectively.

17. The laryngoscopy device of claim 16, wherein said elongated intubation member is a bougie introducer, a stylet, or an endotracheal tube.

18. (canceled)

19. A method for performing laryngoscopy on a subject, comprising the steps of:
providing the laryngoscopy device of any one of the preceding claims and an elongated intubation member;
introducing the laryngoscopy device into the oral cavity of the subject with the looped element being in the first configuration;
maneuvering the laryngoscope blade such as to insert the blade distal portion into the vallecula of the subject, thereby encompassing the epiglottis of the subject with the expanded arch;
introducing the elongated intubation member into the oral cavity and distally advancing the elongated intubation member until a member distal tip thereof passes through the expanded arch and is proximally positioned relative to the corniculate tubercle of the subject;
switching from the first configuration to the second configuration thereby directing the member distal tip towards the laryngeal inlet of the subject; and
distally advancing the member distal tip into the laryngeal inlet of the subject.

20. The method of claim 19, wherein the elongated intubation member is a bougie introducer, and further comprising, following said step of distally advancing the member distal tip into the laryngeal inlet of the subject, a step of mounting an endotracheal tube on the bougie introducer.

21. (canceled)
